(19) United States
(12) Patent Application Publication
(10) Pub. No.: US 2009/0247582 A1
(43) Pub. Date: Oct. 1, 2009

(54) METHODS OF TREATING ATHEROSCLEROSIS

(76) Inventor: Randy Lee Webb, Flemington, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/909,283

(22) PCT Filed: Apr. 25, 2006
(86) PCT No.: PCT/US06/15688

§ 371 Date: Sep. 21, 2007

Related U.S. Application Data
(60) Provisional application No. 60/675,128, filed on Apr. 27, 2005.

(51) Int. Cl. A61K 31/445; A61P 9/10

(52) U.S. Cl. 514/319

(57) ABSTRACT

The present invention relates to a method for the prevention, delay the onset and treatment of atherosclerosis which method comprises administering to a warm-blooded animal, in need thereof, a therapeutically effective amount of a renin inhibitor, or a pharmaceutically acceptable salt thereof, alone or in combination with at least one therapeutic agent selected from the group consisting of:
(1) an ACE inhibitor, or a pharmaceutically acceptable salt thereof;
(2) an angiotensin II receptor blocker, or a pharmaceutically acceptable salt thereof;
(3) a diuretic, or a pharmaceutically acceptable salt thereof;
(4) a calcium channel blocker (CCB), or a pharmaceutically acceptable salt thereof;
(5) a beta-blocker, or a pharmaceutically acceptable salt thereof;
(6) a platelet aggregation inhibitor, or a pharmaceutically acceptable salt thereof;
(7) a cholesterol absorption modulator, or a pharmaceutically acceptable salt thereof;
(8) a HMG-Co-A reductase inhibitor, or a pharmaceutically acceptable salt thereof; and
(9) a high density lipoprotein (HDL) increasing compound, or a pharmaceutically acceptable salt thereof.

Cardiovascular disease is a leading cause of morbidity and mortality, particularly in the United States and in Western European countries. Atherosclerosis, the most prevalent of cardiovascular diseases, is the principle cause of heart attack, stroke and vascular circulation problems. Atherosclerosis is a complex disease which involves many cell types, biochemical events and molecular factors. Several causative factors are implicated in the development of cardiovascular disease including hereditary predisposition to the disease, gender, lifestyle factors such as smoking and diet, age, hypertension, and hyperlipidemia, including hypercholesterolemia. Several of these factors, particularly hyperlipidemia and hypercholesterolemia (high blood cholesterol concentrations) provide a significant risk factor associated with atherosclerosis.

Cholesterol is present in the blood as free and esterified cholesterol within lipoprotein particles, commonly known as chylomicrons, very low density lipoproteins (VLDLs), low density lipoproteins (LDLs), and high density lipoproteins (HDLs). Concentration of total cholesterol in the blood is influenced by (1) absorption of cholesterol from the digestive tract, (2) synthesis of cholesterol from dietary constituents such as carbohydrates, proteins, fats and ethanol, and (3) removal of cholesterol from blood by tissues, especially the liver, and subsequent conversion of the cholesterol to bile acids, steroid hormones, and biliary cholesterol.

Maintenance of blood cholesterol concentrations is influenced by both genetic and environmental factors. Genetic factors include concentration of rate-limiting enzymes in cholesterol biosynthesis, concentration of receptors for low density lipoproteins in the liver, concentration of rate-limiting enzymes for conversion of cholesterols bile acids, rates of synthesis and secretion of lipoproteins and gender of person. Environmental factors influencing the hemostasis of blood cholesterol concentration in humans include dietary composition, incidence of smoking, physical activity, and use of a variety of pharmaceutical agents. Dietary variables include amount and type of fat (saturated and polyunsaturated fatty acids), amount of cholesterol, amount and type of fiber, and perhaps amounts of vitamins such as vitamin C and D and minerals such as calcium.

Clinical studies have firmly established that the elevated plasma concentrations of LDL are associated with accelerated atherogenesis, i.e., formation of atherosclerotic lesions.

It is well understood that hypertension is a leading cause of cardiovascular diseases such as stroke, heart attack, heart failure and irregular heart beat. Hypertension is a condition where the pressure of blood within the blood vessels is higher than normal as it circulates through the body. When the systolic pressure exceeds 150 mmHg or the diastolic pressure exceeds 90 mmHg for a sustained period of time, damage is done to the body. For example, excessive systolic pressure can rupture blood vessels anywhere, and when it occurs within the brain, a stroke results. Hypertension may also cause thickening and narrowing of the blood vessels which ultimately could lead to atherosclerosis.

However, reduction of high blood pressure has an effect on coronary mortality and morbidity lower than expected. One of the possible explanations is the different anti-atherogenic capacity of anti-hypertensive drugs. Reduction of high blood pressure has, by itself, an anti-atherogenic effect, but, for some anti-hypertensive drugs, there is experimental and clinical evidence of anti-atherogenic properties beyond blood pressure lowering, e.g., for calcium antagonists experimental data have been published reporting reduction of aortic lipidic deposition and decrease of arterial proliferation.

Inhibitors of the renin angiotensin system (RAS) are well known drugs that lower blood pressure and exert beneficial actions in hypertension and in congestive heart failure as described, e.g., in N Eng. J. Med. 316: 1429-1435, 1987. The natural enzyme renin is released from the kidneys and cleaves angiotensinogen in the circulation to form the decapeptide angiotensin I. This is in turn cleaved by angiotensin converting enzyme (ACE) in the lungs, kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. Inhibitors of the enzymatic activity of renin bring about a reduction in the formation of angiotensin I. As a result a smaller amount of angiotensin II is produced. The reduced concentration of that active peptide hormone is the direct cause of the antihypertensive effect of renin inhibitors.

However, the role of the renin-angiotensin system in atherosclerosis is not clear. Campbell-Boswell & Robertson Exp. and Mol. Pathol. 35: 265, 1981 reported that angiotensin II stimulated proliferation of isolated human vascular smooth muscle cells while Geisterfer et al. Circ. Res. 62: 749-756, 1988 showed no proliferation of isolated rat vascular smooth muscle cells. Furthermore, Overturf et al. Atherosclerosis, 59: 383-399, 1986, discloses that studies with ACE inhibitors in cholesterol fed rabbits show no significant effects in the development of atherosclerosis.

It has now been surprisingly discovered that renin inhibitors may be employed for the prevention, delay the onset and treatment of atherosclerosis, independent of the antihypertensive effect of renin inhibitors.

Furthermore, the present invention relates to combination therapy administering a renin inhibitor in combination with another therapeutic agent selected from the group consisting of (1) an ACE inhibitor; (2) an angiotensin II receptor blocker; (3) a diuretic; (4) a calcium channel blocker (CCB); (5) a beta-blocker; (6) a platelet aggregation inhibitor; (7) a cholesterol absorption modulator; (8) a HMG-Co-A reductase inhibitor; and (9) a high density lipoprotein (HDL) increasing compound; providing beneficial or synergistic therapeutic effects over each monotherapy component alone.

Accordingly, the present invention further relates to a method for the prevention, delay the onset and treatment of atherosclerosis which method comprises administering to a warm-blooded animal, in need thereof, a therapeutically effective amount of a renin inhibitor, or a pharmaceutically acceptable salt thereof, alone or in combination with at least one therapeutic agent selected from the group consisting of:
(1) an ACE inhibitor, or a pharmaceutically acceptable salt thereof;
(2) an angiotensin II receptor blocker, or a pharmaceutically acceptable salt thereof;
(3) a diuretic, or a pharmaceutically acceptable salt thereof;
(4) a calcium channel blocker (CCB), or a pharmaceutically acceptable salt thereof;
(5) a beta-blocker, or a pharmaceutically acceptable salt thereof;
(6) a platelet aggregation inhibitor, or a pharmaceutically acceptable salt thereof;
(7) a cholesterol absorption modulator, or a pharmaceutically acceptable salt thereof;
(8) a HMG-Co-A reductase inhibitor, or a pharmaceutically acceptable salt thereof; and
(9) a high density lipoprotein (HDL) increasing compound, or a pharmaceutically acceptable salt thereof.

Other objects, features, advantages and aspects of the present invention will become apparent to those skilled in the art from the following description and appended claims. It should be understood, however, that the following description, appended claims, and specific examples, while indicating preferred embodiments of the invention, are given by way of illustration only. Various changes and modifications within the spirit and scope of the disclosed invention will become readily apparent to those skilled in the art from reading the following. Abbreviations are those generally known in the art.

Listed below are some of the definitions of various terms used herein to describe certain aspects of the present invention. However, the definitions used herein are those generally known in the art and apply to the terms as they are used throughout the specification unless they are otherwise limited in specific instances.

The term “prevention” refers to prophylactic administration to healthy patients to prevent the development of the conditions mentioned herein. Moreover, the term “prevention” means prophylactic administration to patients being in a pre-stage of the conditions to be treated.

The term “delay the onset”, as used herein, refers to administration to patients being in a pre-stage of the condition to be treated in which patients with a pre-form of the corresponding condition is diagnosed.

The term “treatment” is understood the management and care of a patient for the purpose of combating the disease, condition or disorder.

The term “therapeutically effective amount” refers to an amount of a drug or a therapeutic agent that will elicit the desired biological or medical response of a tissue, system or an animal (including man) that is being sought by a researcher or clinician.

The term “synergistic”, as used herein, means that the effect achieved with the methods, combinations and pharmaceutical compositions of the present invention is greater than the sum of the effects that result from individual methods and compositions comprising the active ingredients of this invention separately.

The term “warm-blooded animal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art.

The term “combination” of a renin inhibitor, in particular, aliskiren, and another therapeutic agent(s) referred to herein above, or in each case, a pharmaceutically acceptable salt thereof, means that the components can be administered together as a pharmaceutical composition or as part of the same, unitary dosage form. A combination also includes administering a renin inhibitor, in particular, aliskiren, or a pharmaceutically acceptable salt thereof, and another therapeutic agent(s) referred to herein above, or in each case, a pharmaceutically acceptable salt thereof, each separately but as part of the same therapeutic regimen. The components, if administered separately, need not necessarily be administered at essentially the same time, although they can if so desired. Thus, a combination also refers, for example, administering a renin inhibitor, in particular, aliskiren, or a pharmaceutically acceptable salt thereof, and another therapeutic agent(s), or in each case, a pharmaceutically acceptable salt thereof, as separate dosages or dosage forms, but at the same time. A combination also includes separate administration at different times and in any order.

The renin inhibitors to which the present invention applies are any of those having renin inhibitory activity in vivo and, therefore, pharmaceutical utility, e.g., as therapeutic agents for the prevention, delay the onset or treatment of atherosclerosis.

In particular, the present invention relates to renin inhibitors disclosed in U.S. Pat. No. 5,559,111; No. 6,197,959 and No. 6,376,672, the entire contents of which are incorporated herein by reference.

Renin inhibitors include compounds having different structural features. For example, mention may be made of compounds which are selected from the group consisting of ditekiren (chemical name: [1S-[1R*,2R*,4R*(1R*,2R*)]]-1-[(1,1-dimethylethoxy)carbonyl]-L-proly I-L-phenylalanyl-N-[2-hydroxy-5-methyl-1-(2-methylpropyl)-4-[[[2-methyl-1-[[(2-pyridinylmrthyl)amino]carbonyl]butyl]amino]carbonyl]hexyl]-N-alfa-methyl-L-histidinamide); terlakiren (chemical name: [R—(R*,S*)]-N-(4-morpholinylcarbonyl)-L-phenylalanyl-N-[1-(cyclohexylmethyl)-2-hydroxy-3-(1-methylethoxy)-3-oxopropyl]-S-methyl-L-cysteineamide); and zankiren (chemical name: [1S-[1R*[R*(R*)],2S*,3R*]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]-amino]-4-thiazolepropanamide), preferably, in each case, the hydrochloride salt thereof.

Preferred renin inhibitors of the present invention include RO 66-1132 and RO 66-1168 of formulae (I) and (II)

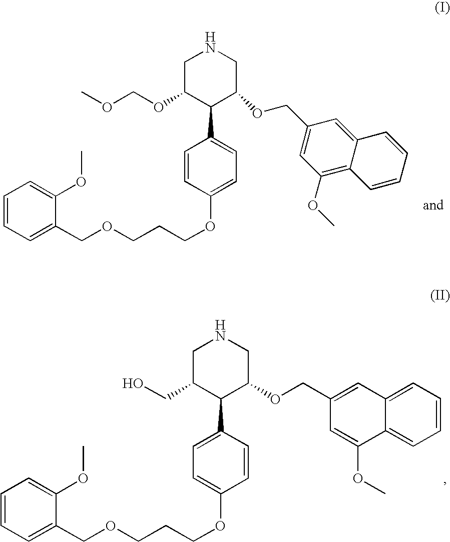

respectively, or a pharmaceutically acceptable salt thereof.

In particular, the present invention relates to a renin inhibitor which is a δ-amino-γ-hydroxy-ω-aryl-alkanoic acid amide derivative of formula (III)

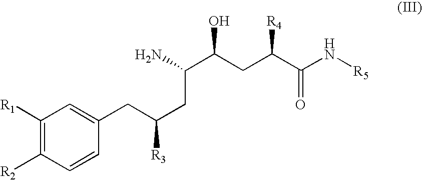

wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

As an alkyl, R₁ may be linear or branched and preferably comprise 1 to 6 C atoms, especially 1 or 4 C atoms. Examples are methyl, ethyl, n- and i-propyl, n-, i- and t-butyl, pentyl and hexyl.

As a halogenalkyl, R₁ may be linear or branched and preferably comprise 1 to 4 C atoms, especially 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

As an alkoxy, R₁ and R₂ may be linear or branched and preferably comprise 1 to 4 C atoms. Examples are methoxy, ethoxy, n- and i-propyloxy, n-, i- and t-butyloxy, pentyloxy and hexyloxy.

As an alkoxyalkyl, R₁ may be linear or branched. The alkoxy group preferably comprises 1 to 4 and especially 1 or 2 C atoms, and the alkyl group preferably comprises 1 to 4 C atoms. Examples are methoxymethyl, 2-methoxyethyl, 3-methoxypropyl, 4-methoxybutyl, 5-methoxypentyl, 6-methoxyhexyl, ethoxymethyl, 2ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

As a C₁₋₆alkoxy-C₁₋₆alkyloxy, R₁ may be linear or branched. The alkoxy group preferably comprises 1 to 4 and especially 1 or 2 C atoms, and the alkyloxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy.

In a preferred embodiment, R₁ is methoxy- or ethoxy-C₁₋₄alkyloxy, and R₂ is preferably methoxy or ethoxy. Particularly preferred are compounds of formula (A), wherein R₁ is 3-methoxypropyloxy and R₂ is methoxy.

As a branched alkyl, R₃ and R₄ preferably comprise 3 to 6 C atoms. Examples are i-propyl, i- and t-butyl, and branched isomers of pentyl and hexyl. In a preferred embodiment, R₃ and R₄ in compounds of formula (A) are in each case i-propyl.

As a cycloalkyl, R₅ may preferably comprise 3 to 8 ring-carbon atoms, 3 or 5 being especially preferred. Some examples are cyclopropyl, cyclobutyl, cyclopentyl, cyclohexyl and cyclooctyl. The cycloalkyl may optionally be substituted by one or more substituents, such as alkyl, halo, oxo, hydroxy, alkoxy, amino, alkylamino, dialkylamino, thiol, alkylthio, nitro, cyano, heterocyclyl and the like.

As an alkyl, R₅ may be linear or branched in the form of alkyl and preferably comprise 1 to 6 C atoms. Examples of alkyl are listed herein above. Methyl, ethyl, n- and i-propyl, n-, i- and t-butyl are preferred.

As a C₁₋₆hydroxyalkyl, R₅ may be linear or branched and preferably comprise 2 to 6 C atoms. Some examples are 2-hydroxyethyl, 2-hydroxypropyl, 3-hydroxypropyl, 2-, 3- or 4-hydroxybutyl, hydroxypentyl and hydroxyhexyl.

As a C₁₋₆alkoxy-C₁₋₆alkyl, R₅ may be linear or branched. The alkoxy group preferably comprises 1 to 4 C atoms and the alkyl group preferably 2 to 4 C atoms. Some examples are 2-methoxyethyl, 2-methoxypropyl, 3-methoxypropyl, 2-, 3- or 4-methoxybutyl, 2-ethoxyethyl, 2-ethoxypropyl, 3-ethoxypropyl, and 2-, 3- or 4-ethoxybutyl.

As a C₁₋₆alkanoyloxy-C₁₋₆alkyl, R₅ may be linear or branched. The alkanoyloxy group preferably comprises 1 to 4 C atoms and the alkyl group preferably 2 to 4 C atoms. Some examples are formyloxymethyl, formyloxyethyl, acetyloxyethyl, propionyloxyethyl and butyroyloxyethyl.

As a C₁₋₆-aminoalkyl, R₅ may be linear or branched and preferably comprise 2 to 4 C atoms. Some examples are 2-aminoethyl, 2- or 3-aminopropyl and 2-, 3- or 4-aminobutyl.

As C₁₋₆alkylamino-C₁₋₆alkyl and C₁₋₆dialkylamino-C₁₋₆alkyl, R₅ may be linear or branched. The alkylamino group preferably comprises C₁₋₄alkyl groups and the alkyl group has preferably 2 to 4 C atoms. Some examples are 2-methylaminoethyl, 2-dimethylaminoethyl, 2-ethylaminoethyl, 2-ethylaminoethyl, 3-methylaminopropyl, 3-dimethylaminopropyl, 4-methylaminobutyl and 4-dimethylaminobutyl.

As a HO(O)C—C₁₋₁₆alkyl, R₅ may be linear or branched and the alkyl group preferably comprises 2 to 4 C atoms. Some examples are carboxymethyl, carboxyethyl, carboxypropyl and carboxybutyl.

As a C₁₋₆alkyl-O—(O)C—C₁₋₆alkyl, R₅ may be linear or branched, and the alkyl groups preferably comprise independently of one another 1 to 4 C atoms. Some examples are methoxycarbonylmethyl, 2-methoxycarbonylethyl, 3-methoxycarbonylpropyl, 4-methoxy-carbonylbutyl, ethoxycarbonylmethyl, 2-ethoxycarbonylethyl, 3-ethoxycarbonylpropyl, and 4-ethoxycarbonylbutyl.

As a H₂N—C(O)—C₁₋₁₆alkyl, R₅ may be linear or branched, and the alkyl group preferably comprises 2 to 6 C atoms. Some examples are carbamidomethyl, 2-carbamidoethyl, 2-carbamido-2,2-dimethylethyl, 2- or 3-carbamidopropyl, 2-, 3- or 4-carbamidobutyl, 3-carbamido-2-methylpropyl, 3-carbamido-1,2-dimethylpropyl, 3-carbamido-3-ethylpropyl, 3-carbamido-2,2-dimethylpropyl, 2-, 3-, 4- or 5-carbamidopentyl, 4-carbamido-3,3- or -2,2-dimethylbutyl. Preferably, R₅ is 2-carbamido-2,2-dimethylethyl.

Accordingly, preferred are δ-amino-γ-hydroxy-ω-aryl-alkanoic acid amide derivatives of formula (III) having the formula

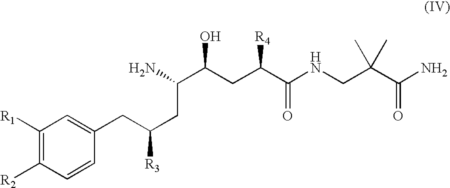

wherein R₁ is 3-methoxypropyloxy; R₂ is methoxy; and R₃ and R₄ are isopropyl; or a pharmaceutically acceptable salt thereof; chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, also known as aliskiren.

The term “aliskiren”, if not defined specifically, is to be understood both as the free base and as a salt thereof, especially a pharmaceutically acceptable salt thereof, most preferably a hemi-fumarate thereof.

Angiotensin II receptor blockers are understood to be those active agents that bind to the AT₁-receptor subtype of angiotensin II receptor but do not result in activation of the receptor. As a consequence of the blockade of the AT₁ receptor, these antagonists can, e.g., be employed as antihypertensive agents.

Suitable angiotensin II receptor blockers which may be employed in the combination of the present invention include AT₁ receptor antagonists having differing structural features, preferred are those with the non-peptidic structures. For example, mention may be made of the compounds that are selected from the group consisting of valsartan (EP 443983), losartan (EP 253310), candesartan (EP 459136), eprosartan (EP 403159), irbesartan (EP 454511), olmesartan (EP 503785), tasosartan (EP 539086), telmisartan (EP 522314), the compound with the designation E-4177 of the formula

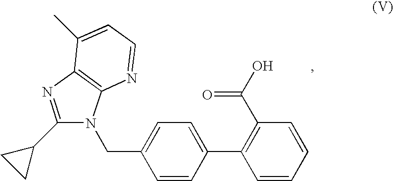

the compound with the designation SC-52458 of the following formula

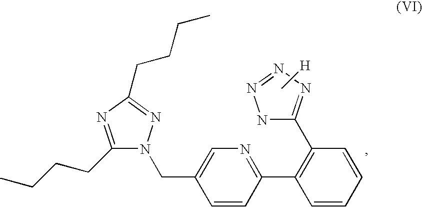

and the compound with the designation the compound ZD-8731 of the formula

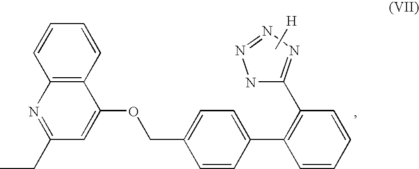

or, in each case, a pharmaceutically acceptable salt thereof.

Preferred AT₁-receptor antagonists are those agents that have reach the market, most preferred is valsartan, or a pharmaceutically acceptable salt thereof.

The interruption of the enzymatic degradation of angiotensin I to angiotensin II with ACE inhibitors is a successful variant for the regulation of blood pressure and thus also makes available a therapeutic method for the treatment of hypertension.

A suitable ACE inhibitor to be employed in the combination of the present invention is, e.g., a compound selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril and trandolapril, or in each case, a pharmaceutically acceptable salt thereof.

Preferred ACE inhibitors are those agents that have been marketed, most preferred are benazepril and enalapril.

A diuretic is, for example, a thiazide derivative selected from the group consisting of chlorothiazide, hydrochlorothiazide, methylclothiazide, and chlorothalidon. The most preferred diuretic is hydrochlorothiazide. A diuretic furthermore is a potassium sparing diuretic such as amiloride or triameterine, or a pharmaceutically acceptable salt thereof.

The class of CCBs essentially comprises dihydropyridines (DHPs) and non-DHPs, such as diltiazem-type and verapamil-type CCBs.

A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g., as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine and verapamil or, e.g., dependent on the specific CCB, a pharmaceutically acceptable salt thereof. Especially preferred as DHP is amlodipine or a pharmaceutically acceptable salt, especially the besylate, thereof. An especially preferred representative of non-DHPs is verapamil or a pharmaceutically acceptable salt, especially the hydrochloride, thereof.

Beta-blockers suitable for use in the present invention include beta-adrenergic blocking agents (beta-blockers) which compete with epinephrine for beta-adrenergic receptors and interfere with the action of epinephrine. Preferably, the beta-blockers are selective for the beta-adrenergic receptor as compared to the alpha-adrenergic receptors, and so do not have a significant alpha-blocking effect. Suitable beta-blockers include compounds selected from acebutolol, atenolol, betaxolol, bisoprolol, carteolol, carvedilol, esmolol, labetalol, metoprolol, nadolol, oxprenolol, penbutolol, pindolol, propranolol, sotalol and timolol. Where the beta-blocker is an acid or base or otherwise capable of forming pharmaceutically acceptable salts or prodrugs, these forms are considered to be encompassed herein, and it is understood that the compounds may be administered in free form or in the form of a pharmaceutically acceptable salt or a prodrug, such as a physiologically hydrolizable and acceptable ester. For example, metoprolol is suitably administered as its tartrate salt, propranolol is suitably administered as the hydrochloride salt, and so forth.

Platelet aggregation inhibitors include PLAVIX® (clopidogrel bisulfate), PLETAL® (cilostazol) and aspirin.

Cholesterol absorption modulators include ZETIA® (ezetimibe) and KT6-971 (Kotobuki Pharmaceutical Co. Japan).

HMG-Co-A reductase inhibitors (also called β-hydroxy-β-methylglutaryl-co-enzyme-A reductase inhibitors or statins) are understood to be those active agents which may be used to lower lipid levels including cholesterol in blood.

The class of HMG-Co-A reductase inhibitors comprises compounds having differing structural features. For example, mention may be made of the compounds which are selected from the group consisting of atorvastatin, cerivastatin, fluvastatin, lovastatin, pitavastatin, pravastatin, rosuvastatin and simvastatin, or in each case, a pharmaceutically acceptable salt thereof.

Preferred HMG-Co-A reductase inhibitors are those agents which have been marketed, most preferred is atorvastatin, pitavastatin or simvastatin, or a pharmaceutically acceptable salt thereof.

HDL increasing compounds include, but are not limited to, cholesterol ester transfer protein (CETP) inhibitors. Examples of CETP inhibitors include JTT705 disclosed in Example 26 of U.S. Pat. No. 6,426,365 issued Jul. 30, 2002, and pharmaceutically acceptable salts thereof.

Preferably, a combination according to the present invention comprises a renin inhibitor, e.g., aliskiren, especially in the form of the hemi-fumarate salt thereof, and an angiotensin II antagonist, e.g., valsartan, or a pharmaceutically acceptable salt thereof, and optionally, a diuretic, e.g., hydrochlorothiazide, or a pharmaceutically acceptable salt thereof, and/or a HMG-Co-A reductase inhibitor, e.g., atorvastatin, pitavastatin and simvastatin, or a pharmaceutically acceptable salt thereof.

Preferred is also a combination according to the present invention which comprises a renin inhibitor, e.g., aliskiren, especially in the form of the hemi-fumarate salt thereof, and an ACE inhibitor, e.g., benazepril or enalapril, or a pharmaceutically acceptable salt thereof.

Preferred is also a combination according to the present invention which comprises a renin inhibitor, e.g., aliskiren, especially in the form of the hemi-fumarate salt thereof, and a diuretic, e.g., hydrochlorothiazide, or a pharmaceutically acceptable salt thereof.

Preferred is also a combination according to the present invention which comprises a renin inhibitor, e.g., aliskiren, especially in the form of the hemi-fumarate salt thereof, and a CCB, e.g., amlodipine, or a pharmaceutically acceptable salt thereof.

Preferred is also a combination according to the present invention which comprises a renin inhibitor, e.g., aliskiren, especially in the form of the hemi-fumarate salt thereof, and a beta-blocker, e.g., acebutolol, atenolol, betaxolol, bisoprolol, carteolol, carvedilol, esmolol, labetalol, metoprolol, nadolol, oxprenolol, penbutolol, pindolol, propranolol, sotalol and timolol, or a pharmaceutically acceptable salt thereof.

Preferred is also a combination according to the present invention which comprises a renin inhibitor, e.g., aliskiren, especially in the form of the hemi-fumarate salt thereof, and a platelet aggregation inhibitor, e.g., clopidogrel or aspirin, or a pharmaceutically acceptable salt thereof.

Preferred is also a combination according to the present invention which comprises a renin inhibitor, e.g., aliskiren, especially in the form of the hemi-fumarate salt thereof, and a HMG-Co-A reductase inhibitor, e.g., atorvastatin, pitavastatin and simvastatin, or a pharmaceutically acceptable salt thereof.

The structure of the active agents identified by generic or tradenames may be taken from the actual edition of the standard compendium “The Merck Index” or the Physician's Desk Reference or from databases, e.g. Patents International (e.g. IMS World Publications) or Current Drugs. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

As referred to herein above, the renin inhibitors of the present invention, and the combination partners thereof, may be present as their pharmaceutically acceptable salts. If these compounds have, e.g., at least one basic center such as an amino group, they can form acid addition salts thereof. Similarly, the compounds having at least one acid group (for example COOH) can form salts with bases. Corresponding internal salts may furthermore be formed, if a compound comprises, e.g., both a carboxy and an amino group.

The corresponding active ingredients or a pharmaceutically acceptable salts may also be used in form of a solvate, such as a hydrate or including other solvents used, e.g., in their crystallization.

In another aspect, the present invention relates to pharmaceutical compositions comprising a renin inhibitor, or a pharmaceutically acceptable salt thereof, preferably aliskiren in the form of the hemi-fumarate salt thereof, and a pharmaceutically acceptable carrier, for the prevention, delay the onset and treatment of atherosclerosis.

Furthermore, the present invention provides pharmaceutical compositions comprising a renin inhibitor, or a pharmaceutically acceptable salt thereof, preferably aliskiren in the form of the hemi-fumarate salt thereof, and at least one therapeutic agent selected from the group consisting of:
(1) an ACE inhibitor, preferably benazepril or enalapril, or in each case, a pharmaceutically acceptable salt thereof;
(2) an angiotensin II receptor blocker, preferably valsartan, or a pharmaceutically acceptable salt thereof;
(3) a diuretic, preferably hydrochlorothiazide, or a pharmaceutically acceptable salt thereof;
(4) a calcium channel blocker (CCB), preferably amlodipine, or a pharmaceutically acceptable salt thereof;
(5) a beta-blocker, or a pharmaceutically acceptable salt thereof;
(6) a platelet aggregation inhibitor, or a pharmaceutically acceptable salt thereof;
(7) a cholesterol absorption modulator, or a pharmaceutically acceptable salt thereof;
(8) a HMG-Co-A reductase inhibitor, preferably atorvastatin, pitavastatin or simvastatin, or in each case, a pharmaceutically acceptable salt thereof; and
(9) a high density lipoprotein (HDL) increasing compound, or a pharmaceutically acceptable salt thereof;
   and a pharmaceutically acceptable carrier; for the prevention, delay the onset and treatment of atherosclerosis.

As disclosed herein above, a renin inhibitor, in particular, aliskiren, preferably in the form of the hemi-fumarate salt thereof, alone or in combination with (1) an ACE inhibitor, e.g., benazepril or enalapril; (2) an angiotensin II receptor blocker, e.g., valsartan; (3) a diuretic, e.g., hydrochlorothiazide; (4) a calcium channel blocker (CCB), e.g., amlodipine; (5) a beta-blocker, e.g., metoprolol; (6) a platelet aggregation inhibitor; (7) a cholesterol absorption modulator; (8) a HMG-Co-A reductase inhibitor, e.g., atorvastatin, pitavastatin or simvastatin; or (9) a high density lipoprotein (HDL) increasing compound; may be co-administered as a pharmaceutical composition. The components may be administered together in any conventional dosage form, usually also together with a pharmaceutically acceptable carrier or diluent.

In carrying out the method of the present invention, the renin inhibitors of the present invention, or the combination partners thereof, may be formulated into pharmaceutical compositions suitable for administration via a variety of routes, such as oral or rectal, transdermal and parenteral administration to mammals, including man. For oral administration the pharmaceutical composition comprising a renin inhibitor, in particular, aliskiren, preferably in the form of the hemi-fumarate salt thereof, or a combination partner thereof, can take the form of solutions, suspensions, tablets, pills, capsules, powders, microemulsions, unit dose packets and the like. Preferred are tablets and gelatin capsules comprising the active ingredient together with: a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-90%, preferably about 1-80%, of the active ingredient.

The dosage of the active ingredients can depend on a variety of factors, such as mode of administration, homeothermic species, age and/or individual condition.

Preferred dosages for the active ingredients of the pharmaceutical combinations according to the present invention are therapeutically effective dosages, especially those which are commercially available.

Normally, in the case of oral administration, an approximate daily dose of from about 1 mg to about 360 mg is to be estimated, e.g., for a patient of approximately 75 kg in weight.

For example, the doses of aliskiren to be administered to warm-blooded animals, including man, of approximately 75 kg body weight, especially the doses effective for the inhibition of renin activity, e.g., in lowering blood pressure, are from about 3 mg to about 3 g, preferably from about 10 mg to about 1 g, e.g., from 20 to 200 mg/person/day, divided preferably into 1 to 4 single doses which may, e.g., be of the same size. Usually, children receive about half of the adult dose. The dose necessary for each individual can be monitored, e.g., by measuring the serum concentration of the active ingredient, and adjusted to an optimum level. Single doses comprise, e.g., 75 mg, 150 mg or 300 mg per adult patient.

In case of ACE inhibitors, preferred dosage unit forms of ACE inhibitors are, for example, tablets or capsules comprising e.g. from about 5 mg to about 20 mg, preferably 5 mg, 10 mg, 20 mg or 40 mg, of benazepril; from about 6.5 mg to 100 mg, preferably 6.25 mg, 12.5 mg, 25 mg, 50 mg, 75 mg or 100 mg, of captopril; from about 2.5 mg to about 20 mg, preferably 2.5 mg, 5 mg, 10 mg or 20 mg, of enalapril; from about 10 mg to about 20 mg, preferably 10 mg or 20 mg, of fosinopril; from about 2.5 mg to about 4 mg, preferably 2 mg or 4 mg, of perindopril; from about 5 mg to about 20 mg, preferably 5 mg, 10 mg or 20 mg, of quinapril; or from about 1.25 mg to about 5 mg, preferably 1.25 mg, 2.5 mg, or 5 mg, of ramipril. Preferred is t.i.d. administration.

Angiotensin II receptor blockers, e.g., valsartan, are supplied in the form of a suitable dosage unit form, e.g., a capsule or tablet, and comprising a therapeutically effective amount of an angiotensin II receptor blocker, e.g., from about 20 to about 320 mg of valsartan, which may be applied to patients. The application of the active ingredient may occur up to three times a day, starting, e.g., with a daily dose of 20 mg or 40 mg of an angiotensin II receptor blocker, e.g., valsartan, increasing via 80 mg daily and further to 160 mg daily, and finally up to 320 mg daily. Preferably, an angiotensin II receptor blocker, e.g., valsartan is applied once a day or twice a day with a dose of 80 mg or 160 mg, respectively, each. Corresponding doses may be taken, e.g., in the morning, at mid-day or in the evening.

In case of diuretics, preferred dosage unit forms are, e.g., tablets or capsules comprising, e.g., from about 5 mg to about 50 mg, preferably from about 6.25 mg to about 25 mg. A daily dose of 6.25 mg, 12.5 mg or 25 mg of hydrochlorothiazide is preferably administered once a day.

In case of CCBs, e.g., amlodipine, preferred dosage unit forms are, e.g., tablets or capsules comprising, e.g., from about 1 mg to about 40 mg, preferably 2.5 to 20 mg daily when administered orally.

In case of HMG-Co-A reductase inhibitors, preferred dosage unit forms of HMG-Co-A reductase inhibitors are, e.g., tablets or capsules comprising e.g. from about 5 mg to about 120 mg, preferably, when using fluvastatin, e.g., 20 mg, 40 mg or 80 mg (equivalent to the free acid) of fluvastatin, e.g., administered once a day.

The above doses encompass a therapeutically effective amount of the active ingredients of the present invention.

Since the present invention relates to methods for the prevention, delay the onset and treatment of with a combination of compounds which may be administered separately, the invention also relates to combining separate pharmaceutical compositions in a kit form. The kit may comprise, e.g., two separate pharmaceutical compositions: (1) a composition comprising a renin inhibitor, in particular, aliskiren, or a pharmaceutically acceptable salt thereof, and a pharmaceutically acceptable carrier or diluent; and (2) a composition comprising another therapeutic agent selected from the group consisting of an ACE inhibitor, an angiotensin II receptor blocker, a diuretic, a calcium channel blocker (CCB), a beta-blocker, a platelet aggregation inhibitor, a cholesterol absorption modulator, a HMG-Co-A reductase inhibitor and a high density lipoprotein (HDL) increasing compound, or in each case, a pharmaceutically acceptable salt thereof, and a pharmaceutically acceptable carrier or diluent. The amounts of (1) and (2) are such that, when co-administered separately a beneficial therapeutic effect(s) is achieved. The kit comprises a container for containing the separate compositions such as a divided bottle or a divided foil packet, wherein each compartment contains a plurality of dosage forms (e.g., tablets) comprising, e.g., (1) or (2). Alternatively, rather than separating the active ingredient-containing dosage forms, the kit may contain separate compartments each of which contains a whole dosage which in turn comprises separate dosage forms. An example of this type of kit is a blister pack wherein each individual blister contains two (or more) tablets, one (or more) tablets) comprising a pharmaceutical composition (1), and the second (or more) tablet(s) comprising a pharmaceutical composition (2). Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician. In the case of the instant invention a kit therefore comprises:

(1) a therapeutically effective amount of a composition comprising a renin inhibitor, in particular, aliskiren, preferably in the form of the hemi-fumarate salt thereof, and a pharmaceutically acceptable carrier or diluent, in a first dosage form;
(2) a composition comprising another therapeutic agent selected from the group consisting of an ACE inhibitor, an angiotensin II receptor blocker, a diuretic, a calcium channel blocker (CCB), a beta-blocker, a platelet aggregation inhibitor, a cholesterol absorption modulator, a HMG-Co-A reductase inhibitor and a high density lipoprotein (HDL) increasing compound, or in each case, a pharmaceutically acceptable salt thereof, in an amount such that, following administration, a beneficial therapeutic effect(s) is achieved, and a pharmaceutically acceptable carrier or diluent, in a second dosage form; and
(3) a container for containing said first and second dosage forms. The action of a renin inhibitor, e.g., aliskiren, alone or in combination with (1) an ACE inhibitor; (2) an angiotensin II receptor blocker; (3) a diuretic; (4) a calcium channel blocker (CCB); (5) a beta-blocker; (6) a platelet aggregation inhibitor; (7) a cholesterol absorption modulator; (8) a HMG-Co-A reductase inhibitor; or (9) a high density lipoprotein (HDL) increasing compound, may be demonstrated inter alia experimentally by means of in vitro tests, e.g., the reduction in the formation of angiotensin I being measured in various systems (human plasma, purified human renin together with synthetic or natural renin substrate).

A renin inhibitor, e.g., aliskiren, or a pharmaceutical salt thereof, or the combination partners thereof, can be administered by various routes of administration. Each agent can be tested over a wide-range of dosages to determine the optimal drug level for each therapeutic agent alone, or in the specific combination thereof, to elicit the maximal response. For these studies, it is preferred to use treatment groups consisting of at least 6 animals per group. Each study is best performed in away wherein the effects of the combination treatment group are determined at the same time as the individual components are evaluated. Although drug effects may be observed with acute administration, it is preferable to observe responses in a chronic setting. The long-term study is of sufficient duration to allow for the full development of compensatory responses to occur and, therefore, the observed effect will most likely depict the actual responses of the test system representing sustained or persistent effects.

Representative studies may be carried out, e.g., with aliskiren, employing an apolipoprotein E knockout mouse model which has become one of the primary models for atherosclerosis (Arterioscler Thromb Vasc Biol., 24: 1006-1014, 2004; Trends Cardiovasc Med, 14: 187-190, 2004). The studies may be performed as described by Johnson et al. in Circulation, 111: 1422-1430, 2005, or using modifications thereof.

The available results indicate that renin inhibitors may be employed for the prevention, delay the onset and treatment of atherosclerosis, independent of the antihypertensive effect of renin inhibitors.

Furthermore, it has been found that, a combination of a renin inhibitor, e.g., aliskiren, especially in the form of the hemi-fumarate salt thereof, with (1) an ACE inhibitor; (2) an angiotensin II receptor blocker; (3) a diuretic; (4) a calcium channel blocker (CCB); (5) a beta-blocker; (6) a platelet aggregation inhibitor; (7) a cholesterol absorption modulator; (8) a HMG-Co-A reductase inhibitor; or (9) a high density lipoprotein (HDL) increasing compound, or in each case, a pharmaceutically acceptable salt thereof, achieves greater therapeutic effect than the administration of a renin inhibitor alone. Greater efficacy can also be documented as a prolonged duration of action. The duration of action can be monitored as either the time to return to baseline prior to the next dose or as the area under the curve (AUC).

Further benefits are that lower doses of the individual drugs to be combined according to the present invention can be used to reduce the dosage, e.g., that the dosages need not only often be smaller but are also applied less frequently, or can be used to diminish the incidence of side effects. The combined administration of a renin inhibitor, e.g., aliskiren, or a pharmaceutically acceptable salt thereof, with (1) an ACE inhibitor; (2) an angiotensin II receptor blocker; (3) a diuretic; (4) a calcium channel blocker (CCB); (5) a beta-blocker; (6) a platelet aggregation inhibitor; (7) a cholesterol absorption modulator; (8) a HMG-Co-A reductase inhibitor; or (9) a high density lipoprotein (HDL) increasing compound, or in each case, a pharmaceutically acceptable salt thereof, results in a significant response in a greater percentage of treated patients, i.e., a greater responder rate results.

It can be shown that combination therapy with a renin inhibitor, e.g., aliskiren, especially in the form of the hemi-fumarate salt thereof, and (1) an ACE inhibitor; (2) an angiotensin II receptor blocker; (3) a diuretic; (4) a calcium channel blocker (CCB); (5) a beta-blocker; (6) a platelet aggregation inhibitor; (7) a cholesterol absorption modulator; (8) a HMG-Co-A reductase inhibitor; or (9) a high density lipoprotein (HDL) increasing compound, or in each case, a pharmaceutically acceptable salt thereof, results in a more effective therapy for the prevention, delay the onset or the treatment of atherosclerosis. In particular, all the more surprising is the experimental finding that a combination of the present invention results in a beneficial, especially a synergistic, therapeutic effect but also in benefits resulting from combined treatment such as a surprising prolongation of efficacy.

The invention furthermore relates to the use of a renin inhibitor, e.g., aliskiren, alone or in combination with (1) an ACE inhibitor; (2) an angiotensin II receptor blocker; (3) a diuretic; (4) a calcium channel blocker (CCB); (5) a beta-blocker; (6) a platelet aggregation inhibitor; (7) a cholesterol absorption modulator; (8) a HMG-Co-A reductase inhibitor; or (9) a high density lipoprotein (HDL) increasing compound, or in each case, a pharmaceutically acceptable salt thereof, for the manufacture of a medicament for the prevention of, delay the onset or the treatment of atherosclerosis.

Accordingly, another embodiment of the present invention relates to the use of a renin inhibitor, e.g., aliskiren, alone or in combination with (1) an ACE inhibitor, or a pharmaceutically acceptable salt thereof; (2) an angiotensin II receptor blocker, or a pharmaceutically acceptable salt thereof; (3) a diuretic, or a pharmaceutically acceptable salt thereof; (4) a calcium channel blocker (CCB), or a pharmaceutically acceptable salt thereof; (5) a beta-blocker, or a pharmaceutically acceptable salt thereof; (6) a platelet aggregation inhibitor, or a pharmaceutically acceptable salt thereof; (7) a cholesterol absorption modulator, or a pharmaceutically acceptable salt thereof; (8) a HMG-Co-A reductase inhibitor, or a pharmaceutically acceptable salt thereof; or (9) a high density lipoprotein (HDL) increasing compound, or in each case, a pharmaceutically acceptable salt thereof, for the manufacture of a medicament for the prevention of, delay the onset or the treatment of atherosclerosis.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative of certain aspects of the present invention and are not a limitation of the scope of the present invention in any way.

### EXAMPLE 1

### Composition of Aliskiren 150 Mg (Free Base) Uncoated Tablets in Mg/Unit

| | Roller | | | |
|---|---|---|---|---|
| | compacted | Dosage | Dosage | Dosage form |
| Component | tablet | form 1 | form 2 | 3 |
| Aliskiren hemi-fumarate | 165.750 | 165.750 | 165.750 | 165.750 |
| Microcrystalline cellulose | 220.650 | 84.750 | 72.250 | 107.250 |
| Polyvinylpyrrolidon K 30 | — | — | 12.000 | 12.000 |
| Crospovidone | 84.000 | 45.000 | 44.000 | 48.200 |
| Aerosil 200 | 4.800 | 1.500 | 1.500 | 1.800 |
| Magnesium stearate | 4.800 | 3.000 | 4.500 | 5.000 |
| Total weight | 480.000 | 300.000 | 300.000 | 340.000 |

### Composition of Aliskiren 150 Mg (Free Base) Uncoated Tablets in % by Weight.

| | Roller | | | |
|---|---|---|---|---|
| | compacted | Dosage | Dosage | Dosage form |
| Component | tablet | form 1 | form 2 | 3 |
| Aliskiren hemi-fumarate | 34.53 | 55.25 | 55.25 | 48.75 |
| Microcrystalline cellulose | 45.97 | 28.25 | 24.08 | 31.545 |
| Polyvinylpyrrolidon K 30 | — | — | 4 | 3.53 |
| Crospovidone | 17.5 | 15 | 14.67 | 14.175 |
| Aerosil 200 | 1 | 0.5 | 0.5 | 0.53 |
| Magnesium stearate | 1 | 1 | 1.5 | 1.47 |
| Total % | 100.00 | 100.00 | 100.00 | 100.00 |

Composition of Aliskiren 150 Mg (Free Base) Uncoated Tablets in Mg/Unit (Divided into Inner/Outer Phase).

| | | Roller | | | |
|---|---|---|---|---|---|
| | | compacted | | | |
| | Component | tablet | Dosage form 1 | Dosage form 2 | Dosage form 3 |
| Inner | Aliskiren hemi-fumarate | 165.75 | 165.75 | 165.75 | 165.75 |
| Phase | Microcrystalline cellulose | 220.65 | 84.75 | 72.25 | 90.25 |
| | Polyvinylpyrrolidon K 30 | — | — | 12.00 | 12.00 |
| | Crospovidone | 36.00 | — | — | 14.20 |
| | Aerosil 200 | — | — | — | — |
| | Magnesium stearate | 2.40 | — | — | — |
| Outer | Crospovidone | 48.00 | 45.00 | 44.00 | 34.00 |
| phase | Microcrystalline cellulose | — | — | — | 17.00 |
| | Aerosil 200 | 4.80 | 1.50 | 1.50 | 1.80 |
| | Magnesium stearate | 2.40 | 3.00 | 4.50 | 5.00 |
| | Total weight | 480.00 | 300.00 | 300.00 | 340.00 |

Composition of Aliskiren 150 Mg (Free Base) Uncoated Tablets in % by Weight (Divided into Inner/Outer Phase).

| | | Roller | | | |
|---|---|---|---|---|---|
| | | compacted | | | |
| | Component | tablet | Dosage form 1 | Dosage form 2 | Dosage form 3 |
| Inner | Aliskiren hemi-fumarate | 34.53 | 55.25 | 55.25 | 48.75 |
| Phase | Microcrystalline cellulose | 45.97 | 28.25 | 24.08 | 26.545 |
| | Polyvinylpyrrolidon K 30 | — | — | 4 | 3.530 |
| | Crospovidone | 7.5 | — | — | 4.175 |
| | Aerosil 200 | — | — | — | — |
| | Magnesium stearate | 0.5 | — | — | — |
| Outer | Crospovidone | 10 | 15 | 14.67 | 10 |
| phase | Microcrystalline cellulose | — | — | — | 5 |
| | Aerosil 200 | 1 | 0.5 | 0.5 | 0.53 |
| | Magnesium stearate | 0.5 | 1 | 1.5 | 1.47 |
| | Total % | 100.00 | 100.00 | 100.00 | 100.00 |

### EXAMPLE 2

### Composition of Aliskiren (Dosage Form 3) Film-Coated Tablets in Mg/Unit

| | Dosage form 3/Strength | | |
|---|---|---|---|
| | 75 mg | 150 mg | 300 mg |
| Component | (free base) | (free base) | (free base) |
| Aliskiren hemi-fumarate | 82.875 | 165.750 | 331.500 |
| Microcrystalline cellulose | 53.625 | 107.250 | 214.500 |
| Polyvinylpyrrolidon K 30 | 6.000 | 12.000 | 24.000 |
| Crospovidone | 24.100 | 48.200 | 96.400 |
| Aerosil 200 | 0.900 | 1.800 | 3.600 |
| Magnesium stearate | 2.500 | 5.000 | 10.000 |
| Total tablet weight | 170.000 | 340.000 | 680.000 |
| Opadry premix white | 9.946 | 16.711 | 23.9616 |
| Opadry premix red | 0.024 | 0.238 | 1.8382 |
| Opadry premix black | 0.030 | 0.051 | 0.2002 |
| Total fim-coated tablet | 180.000 | 357.000 | 706.000 |
| weight | | | |

The dosages forms 1, 2 and 3 may be prepared, e.g., as follows:
1) mixing the active ingredient and additives and granulating said components with a granulation liquid;
2) drying a resulting granulate;
3) mixing the dried granulate with outer phase excipients;
4) compressing a resulting mixture to form a solid oral dosage as a core tablet; and
5) optionally coating a resulting core tablet to give a film-coated tablet.

The granulation liquid can be ethanol, a mixture of ethanol and water, a mixture of ethanol, water and isopropanol, or a solution of polyvinylpyrrolidones (PVP) in the before mentioned mixtures. A preferred mixture of ethanol and water ranges from about 50/50 to about 99/1 (% w/w), most preferrably it is about 94/6 (% w/w). A preferred mixture of ethanol, water and isopropanol ranges from about 45/45/5 to about 98/1/1 (% w/w/w), most preferably from about 88.5/5.5/6.0 to about 91.5/4.5/4.0 (% w/w/w). A preferred concentration of PVP in the above named mixtures ranges from about 5 to about 30% by weight, preferably from about 15 to about 25%, more preferably from about 16 to about 22%.

Attention is drawn to the numerous known methods of granulating, drying and mixing employed in the art, e.g., spray granulation in a fluidized bed, wet granulation in a high-shear mixer, melt granulation, drying in a fluidized-bed dryer, mixing in a free-fall or tumble blender, compressing into tablets on a single-punch or rotary tablet press.

The manufacturing of the granulate can be performed on standard equipment suitable for organic granulation processes. The manufacturing of the final blend and the compression of tablets can also be performed on standard equipment.

For example, step (1) may be carried out by a high-shear granulator, e.g., Collette Gral; step (2) may be conducted in a fluid-bed dryer; step (3) may be carried out by a free-fall mixer (e.g. container blender, tumble blender); and step (4) may be carried out using a dry compression method, e.g., a rotary tablet press.

### EXAMPLE 3

### Film-Coated Tablets

| | Composition Per | |
|---|---|---|
| Components | Unit (mg) | Standards |
| Granulation | | |
| Valsartan [=active ingredient] | 80.00 | |
| Microcrystalline cellulose/ | 54.00 | NF, Ph. Eur |
| Avicel PH 102 | | |
| Crospovidone | 20.00 | NF, Ph. Eur |
| Colloidal anhydrous silica/ | 0.75 | Ph. Eur/NF |
| colloidal silicon dioxide/Aerosil 200 | | |
| Magnesium stearate | 2.5 | NF, Ph. Eur |
| Blending | | |
| Colloidal anhydrous silica/ | 0.75 | Ph. Eur/NF |
| colloidal silicon dioxide/Aerosil 200 | | |
| Magnesium stearate | 2.00 | NF, Ph. Eur |
| Coating | | |
| Purified water*⁾ | — | |
| DIOLACK pale red 00F34899 | 7.00 | |
| Total tablet mass | 167.00 | |

*⁾Removed during processing.

The film-coated tablets may be manufactured, e.g., as follows:

A mixture of valsartan, microcrystalline cellulose, crospovidone, part of the colloidal anhydrous silica/colloidal silicon dioxide/Aerosile 200, silicon dioxide and magnesium stearate is premixed in a diffusion mixer and then sieve through a screening mill. The resulting mixture is again pre-mixed in a diffusion mixer, compacted in a roller compactor and then sieve through a screening mill. To the resulting mixture, the rest of the colloidal anhydrous silica/colloidal silicon dioxide/Aerosile 200 are added and the final blend is made in a diffusion mixer. The whole mixture is compressed in a rotary tabletting machine and the tablets are coated with a film by using Diolack pale red in a perforated pan.

### EXAMPLE 4

### Film-Coated Tablets

| | Composition Per | |
|---|---|---|
| Components | Unit (mg) | Standards |
| Granulation | | |
| Valsartan [=active ingredient] | 160.00 | |
| Microcrystalline cellulose/ | 108.00 | NF, Ph. Eur |
| Avicel PH 102 | | |
| Crospovidone | 40.00 | NF, Ph. Eur |
| Colloidal anhydrous silica/ | 1.50 | Ph. Eur/NF |
| colloidal silicon dioxide/Aerosil 200 | | |
| Magnesium stearate | 5.00 | NF, Ph. Eur |
| Blending | | |
| Colloidal anhydrous silica/ | 1.50 | Ph. Eur/NF |
| colloidal silicon dioxide/Aerosil 200 | | |
| Magnesium stearate | 4.00 | NF, Ph. Eur |
| Coating | | |
| Opadry Light Brown 00F33172 | 10.00 | |
| Total tablet mass | 330.00 | |

The film-coated tablets are manufactured, e.g., as described in Example 3.

### EXAMPLE 5

### Film-Coated Tablets

| | Composition Per | |
|---|---|---|
| Components | Unit (mg) | Standards |
| Core: Internal phase | | |
| Valsartan | 40.00 | |
| [=active ingredient] | | |
| Silica, colloidal anhydrous | 1.00 | Ph. Eur, USP/NF |
| (Colloidal silicon dioxide) | | |
| [=Glidant] | | |
| Magnesium stearate | 2.00 | USP/NF |
| [=Lubricant] | | |
| Crospovidone | 20.00 | Ph. Eur |
| [Disintegrant] | | |
| Microcrystalline cellulose | 124.00 | USP/NF |
| [=Binding agent] | | |
| External phase | | |
| Silica, colloidal anhydrous, | 1.00 | Ph. Eur, USP/NF |
| (Colloidal silicon dioxide) | | |
| [=Glidant] | | |
| Magnesium stearate | 2.00 | USP/NF |
| [Lubricant] | | |
| Film coating | | |
| Opadry ® brown OOF 16711*⁾ | 9.40 | |
| Purified Water**⁾ | — | |
| Total tablet mass | 199.44 | |

*⁾The composition of the Opadry ® brown OOF16711 coloring agent is tabulated below.
**⁾Removed during processing.

### Opadry® Composition:

| | Approximate |
|---|---|
| Ingredient | % Composition |
| Iron oxide, black (C.I. No. 77499, E 172) | 0.50 |
| Iron oxide, brown (C.I. No. 77499, E 172 | 0.50 |
| Iron oxide, red (C.I. No. 77491, E 172) | 0.50 |
| Iron oxide, yellow (C.I. No. 77492, E 172) | 0.50 |
| Macrogolum (Ph. Eur) | 4.00 |
| Titanium dioxide (C.I. No. 77891, E 171) | 14.00 |
| Hypromellose (Ph. Eur) | 80.00 |

The film-coated tablets are manufactured, e.g., as described in Example 3.

### EXAMPLE 6

### Capsules

| | Composition Per Unit |
|---|---|
| Components | (mg) |
| Valsartan [=active ingredient] | 80.00 |
| Microcrystalline cellulose | 25.10 |
| Crospovidone | 13.00 |
| Povidone | 12.50 |
| Magnesium stearate | 1.30 |
| Sodium lauryl sulphate | 0.60 |
| Shell | |
| Iron oxide, red | 0.123 |
| (C.I. No. 77491, EC No. E 172) | |
| Iron oxide, yellow | 0.123 |
| (C.I. No. 77492, EC No. E 172) | |
| Iron oxide, black | 0.245 |
| (C.I. No. 77499, EC No. E 172) | |
| Titanium dioxide | 1.540 |
| Gelatin | 74.969 |
| Total mass | 209.50 |

The capsules may be manufactured, e.g., as follows:

### Granulation/Drying:

Valsartan and microcrystallin cellulose are spray-granulated in a fluidized bed granulator with a granulating solution consisting of povidone and sodium lauryl sulphate dissolved in purified water. The granulate obtained is dried in a fluidized bed dryer.

### Milling/Blending:

The dried granulate is milled together with crospovidone and magnesium stearate. The mass is then blended in a conical srew type mixer for approximately 10 minutes.

### Encapsulation:

The empty hard gelatin capsules are filled with the blended bulk granules under controlled temperature and humidity conditions. The filed capsules are dedusted, visually inspected, weightchecked and quarantined until by Quality assurance department.

### EXAMPLE 7

### Capsules

| Components | Composition Per Unit (mg) |
|---|---|
| Valsartan [=active ingredient] | 160.00 |
| Microcrystalline cellulose | 50.20 |
| Crospovidone | 26.00 |
| Povidone | 25.00 |
| Magnesium stearate | 2.60 |
| Sodium lauryl sulphate | 1.20 |
| Shell | |
| Iron oxide, red | 0.123 |
| (C.I. No. 77491, EC No. E 172) | |
| Iron oxide, yellow | 0.123 |
| (C.I. No. 77492, EC No. E 172) | |
| Iron oxide, black | 0.245 |
| (C.I. No. 77499, EC No. E 172) | |
| Titanium dioxide | 1.540 |
| Gelatin | 74.969 |
| Total mass | 342.00 |

The capsules are manufactured, e.g., as described in Example 6.

### EXAMPLE 8

### Hard Gelatine Capsules

| Components | Composition Per Unit (mg) |
|---|---|
| Valsartan [=active ingredient] | 80.00 |
| Sodium laurylsulphate | 0.60 |
| Magnesium stearate | 1.30 |
| Povidone | 12.50 |
| Crospovidone | 13.00 |
| Microcrystalline cellulose | 21.10 |
| Total mass | 130.00 |

### EXAMPLE 9

### Hard Gelatin Capsules

| Components | Composition Per Unit (mg) |
|---|---|
| Valsartan [=active ingredient] | 80.00 |
| Microcrystalline cellulose | 110.00 |
| Povidone K30 | 45.20 |
| Sodium laurylsulphate | 1.20 |
| Magnesium stearate | 2.60 |
| Crospovidone | 26.00 |
| Total mass | 265.00 |

Components (1) and (2) are granulated with a solution of components (3) and (4) in water. The components (5) and (6) are added to the dry granulate and the mixture is filled into size hard gelatin capsules.

All publications and patents mentioned herein are incorporate by reference in their entirety as if set forth in full herein.

1. A method for the prevention, delay the onset or treatment of atherosclerosis, which method comprises administering to a patient, in need thereof, a therapeutically effective amount of a renin inhibitor, or a pharmaceutically acceptable salt thereof.

2. A method for the prevention, delay the onset or treatment of atherosclerosis, which method comprises administering to a patient, in need thereof, a therapeutically effective amount of a therapeutic agent consisting of a renin inhibitor, or a pharmaceutically acceptable salt thereof.

3. A method according to claim 1, wherein a renin inhibitor is selected from the group consisting of RO 66-1132, RO 66-1168 and a compound of formula (III) 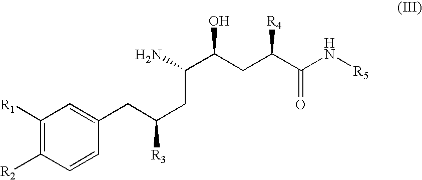
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or in each case a pharmaceutically acceptable salt thereof.

4. A method according to claim 3, wherein a renin inhibitor is a compound of formula (III) having the formula 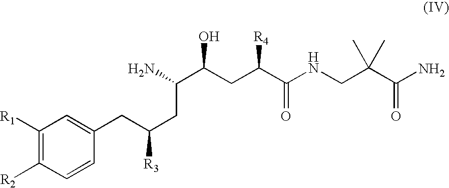
wherein R₁ is 3-methoxypropyloxy; R₂ is methoxy; and R₃ and R₄ are isopropyl; or a pharmaceutically acceptable salt thereof.

5. A method according to claim 4, wherein the compound of formula (IV) is in the form of the hemi-fumarate salt thereof.

6-15. (canceled)

16. A method for the prevention, delay the onset or treatment of atherosclerosis, which method comprises administering to a patient, in need thereof, a therapeutically effective amount a combination of a renin inhibitor, or a pharmaceutically acceptable salt thereof, with at least one therapeutic agent selected from the group consisting of:
(1) an ACE inhibitor, or a pharmaceutically acceptable salt thereof;
(2) an angiotensin II receptor blocker, or a pharmaceutically acceptable salt thereof;
(3) a diuretic, or a pharmaceutically acceptable salt thereof;
(4) a calcium channel blocker (CCB), or a pharmaceutically acceptable salt thereof;
(5) a beta-blocker, or a pharmaceutically acceptable salt thereof;
(6) a platelet aggregation inhibitor, or a pharmaceutically acceptable salt thereof;
(7) a cholesterol absorption modulator, or a pharmaceutically acceptable salt thereof;
(8) a HMG-Co-A reductase inhibitor, or a pharmaceutically acceptable salt thereof; and
(9) a high density lipoprotein (HDL) increasing compound, or a pharmaceutically acceptable salt thereof.

17. A method for the prevention, delay the onset or treatment of atherosclerosis, which method comprises administering to a patient, in need thereof, a therapeutically effective amount a combination of a renin inhibitor, or a pharmaceutically acceptable salt thereof, with at least one therapeutic agent selected from the group consisting of:
(1) a platelet aggregation inhibitor, or a pharmaceutically acceptable salt thereof;
(2) a cholesterol absorption modulator, or a pharmaceutically acceptable salt thereof; and
(3) a high density lipoprotein (HDL) increasing compound, or a pharmaceutically acceptable salt thereof.

18. A method according to claim 16, wherein a renin inhibitor is selected from the group consisting of RO 66-1132, RO 66-1168 and a compound of the formula 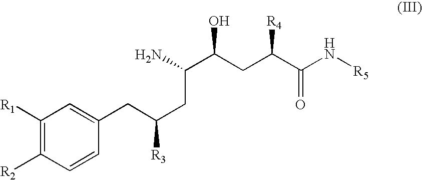
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₁₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

19. A method according to claim 18, wherein a renin inhibitor is a compound of formula (III) having the formula 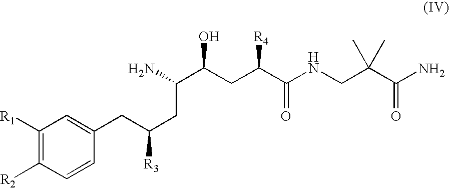
wherein R₁ is 3-methoxypropyloxy; R₂ is methoxy; and R₃ and R₄ are isopropyl; or a pharmaceutically acceptable salt thereof.

20. A method according to claim 19, wherein the compound of formula (IV) is in the form of the hemi-fumarate salt thereof.

21. A method according to claim 16, wherein an ACE inhibitor is selected from the group consisting of benazepril and enalapril, or in each case, a pharmaceutically acceptable salt thereof.

22. A method according to claim 16, wherein an angiotensin II antagonist is valsartan, or a pharmaceutically acceptable salt thereof.

23. A method according to claim 16, wherein a diuretic is hydrochlorothiazide, or a pharmaceutically acceptable salt thereof.

24. A method according to claim 16, wherein a HMG-Co-A reductase inhibitor is selected from the group consisting of atorvastatin, pitavastatin simvastatin, or a pharmaceutically acceptable salt thereof.

25. A method according to claim 16, wherein a calcium channel blocker is amlodipine, or a pharmaceutically acceptable salt thereof.

26. A method according to claim 16, wherein a beta-blocker is selected from the group consisting of acebutolol, atenolol, betaxolol, bisoprolol, carteolol, carvedilol, esmolol, labetalol, metoprolol, nadolol, oxprenolol, penbutolol, pindolol, propranolol, sotalol and timolol, or a pharmaceutically acceptable salt thereof.

27. A method according to claim 16, wherein a platelet aggregation inhibitor is selected from the group consisting of clopidogrel and aspirin, or a pharmaceutically acceptable salt thereof.

28. A method according to claim 16, wherein a HMG-Co-A reductase inhibitor is selected from the group consisting of atorvastatin, pitavastatin and simvastatin, or a pharmaceutically acceptable salt thereof.

29. A pharmaceutical composition comprising a renin inhibitor, or a pharmaceutically acceptable salt thereof, and at least one therapeutic agent selected from the group consisting of:
(1) an ACE inhibitor, or a pharmaceutically acceptable salt thereof;
(2) an angiotensin II receptor blocker, or a pharmaceutically acceptable salt thereof;
(3) a diuretic, or a pharmaceutically acceptable salt thereof;
(4) a calcium channel blocker (CCB), or a pharmaceutically acceptable salt thereof;
(5) a beta-blocker, or a pharmaceutically acceptable salt thereof;
(6) a platelet aggregation inhibitor, or a pharmaceutically acceptable salt thereof;
(7) a cholesterol absorption modulator, or a pharmaceutically acceptable salt thereof;
(8) a HMG-Co-A reductase inhibitor, or a pharmaceutically acceptable salt thereof; and (9) a high density lipoprotein (HDL) increasing compound, or a pharmaceutically acceptable salt thereof;
and a pharmaceutically acceptable carrier; for the prevention of, delay the onset or the treatment of atherosclerosis.

30. A pharmaceutical composition comprising a renin inhibitor, or a pharmaceutically acceptable salt thereof, and at least one therapeutic agent selected from the group consisting of:
(1) a platelet aggregation inhibitor, or a pharmaceutically acceptable salt thereof;
(1) a cholesterol absorption modulator, or a pharmaceutically acceptable salt thereof; and
(3) a high density lipoprotein (HDL) increasing compound, or a pharmaceutically acceptable salt thereof;
and a pharmaceutically acceptable carrier; for the prevention of, delay the onset or the treatment of atherosclerosis.

31. A pharmaceutical composition according to claim 29, wherein a renin inhibitor is selected from the group consisting of RO 66-1132, RO 66-1168 and a compound of the formula 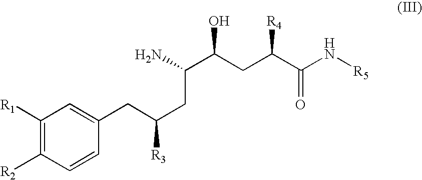
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

32. A pharmaceutical composition according to claim 31, wherein a renin inhibitor is a compound of formula (III) having the formula 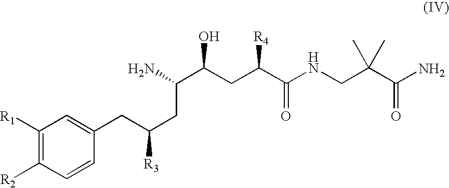
wherein R₁ is 3-methoxypropyloxy; R₂ is methoxy; and R₃ and R₄ are isopropyl; or a pharmaceutically acceptable salt thereof.

33. A pharmaceutical composition according to claim 32, wherein the compound of formula (IV) is in the form of the hemi-fumarate salt thereof.

34. A pharmaceutical composition according to claim 29, wherein an ACE inhibitor is selected from the group consisting of benazepril and enalapril, or in each case, a pharmaceutically acceptable salt thereof.

35. A pharmaceutical composition according to claim 29, wherein an angiotensin II antagonist is valsartan, or a pharmaceutically acceptable salt thereof.

36. A pharmaceutical composition according to claim 29, wherein a diuretic is hydrochlorothiazide, or a pharmaceutically acceptable salt thereof.

37. A pharmaceutical composition according to claim 29, wherein a HMG-Co-A reductase inhibitor is selected from the group consisting of atorvastatin, pitavastatin and simvastatin, or a pharmaceutically acceptable salt thereof.

38. A pharmaceutical composition according to claim 29, wherein a calcium channel blocker is amlodipine, or a pharmaceutically acceptable salt thereof.

39. A pharmaceutical composition according to claim 29, wherein a beta-blocker is selected from the group consisting of acebutolol, atenolol, betaxolol, bisoprolol, carteolol, carvedilol, esmolol, labetalol, metoprolol, nadolol, oxprenolol, penbutolol, pindolol, propranolol, sotalol and timolol, or a pharmaceutically acceptable salt thereof.

40. A pharmaceutical composition according to claim 29, wherein a platelet aggregation inhibitor is selected from the group consisting of clopidogrel and aspirin, or a pharmaceutically acceptable salt thereof.

41. A method according to claim 29, wherein a HMG-Co-A reductase inhibitor is selected from the group consisting of atorvastatin, pitavastatin and simvastatin, or a pharmaceutically acceptable salt thereof.
